(19) United States
(12) Patent Application Publication
(10) Pub. No.: US 2025/0034497 A1
(43) Pub. Date: Jan. 30, 2025

(54) DEVICE FOR PROPAGATING DIFFERENTIATED AND UNDIFFERENTIATED CELLS AND USE OF THE DEVICE FOR THE PRODUCTION OF CULTIVATED FOOD

(71) Applicant: Green Elephant Biotech GmbH, Giessen (DE)

(72) Inventors: Joel EICHMANN, Giessen (DE); Lukas KAESSER, Staufenberg (DE)

(21) Appl. No.: 18/785,316

(22) Filed: Jul. 26, 2024

(30) Foreign Application Priority Data
Jul. 28, 2023 (EP) 23188450.3

(51) Int. Cl. C12M 1/12; C12M 1/00; C12M 1/34

(52) U.S. Cl. CPC C12M 23/04; C12M 23/26; C12M 41/30

(57) ABSTRACT

A device for propagating cells, comprising a housing with a longitudinal orientation extending from a first housing end to a second housing end, a plastic film present in the housing and wound up in a spiral shape around a winding axis oriented along or parallel to the longitudinal orientation, with a first and a second film end and with an inner and an opposite outer side, wherein on the inner side or on the outer side or on the inner and the outer side of the plastic film a multiplicity of spacers is present, arranged and adapted to keep the inner side of the wound up plastic film spaced apart from the outer side in sections or substantially completely in the wound up state, such that a liquid medium can be transferred along the longitudinal orientation. Use of the device for the production of cultivated food or for the production of propagated differentiated or undifferentiated cells.

### FIELD

The present disclosure relates to a device for propagating differentiated and undifferentiated cells. Furthermore, the present disclosure relates to the use of the device according to the present disclosure for propagating differentiated and undifferentiated cells. In addition, the present disclosure relates to the use of the device according to the present disclosure for the production of propagated differentiated as well as undifferentiated cells. Finally, the present disclosure relates to the use of the device according to the present disclosure for the production of cultivated food such as cultivated meat.

### BACKGROUND

Meat-based foods as well as the use of fish and seafood as foods are increasingly questioned by many consumers for a wide variety of reasons. Aspects of mass animal husbandry as well as ethical concerns of fundamental nature and considerations of sustainability play into these considerations. Thus, the contribution of industrial meat production to global warming is not to be underestimated, but herds of cattle worldwide are responsible for a large part of the total methane emissions. Undisputedly, for the production of a kilocalorie from meat a multiple of this nutritional value in the form of feed plants is necessary. The fact that the need for meat-free foods can be met by a steadily growing world population in view of a growing demand for such foods is considered to be critical. For some time, there thus have been attempts to arrive at meat products, so-called cultivated meat or in vitro meat, in an artificial way.

DE 42 00 446 A1 discloses a bioreactor which is equipped with a cell carrier arrangement, with which biomaterial such as cells can be produced in an optimum way. For this purpose, the cell carrier arrangement has to be formed at least double-walled and to consist of at least two blanks of a flat material, wherein the blanks have to form at least one gap or a gap-like region between them.

DE 22 36 240 A1 relates to an apparatus for the cultivation of tissue cells and microorganisms, in which the surface arranged in a container is wound up in a spiral winding on a substantially horizontal, rotatable shaft in such a way that a distance is kept between the windings. In this case, the rotating surface has to be closed off at each of its end sides by a disk against the containers, and the disks have to have openings at the part adjacent to the innermost winding. In this way, the cultivation of tissue cells on a large-area surface is to succeed in a simple way, wherein the tissue cells obtained can be easily harvested.

DE 195 46 542 C1 relates to a cell cultivation vessel with a cell culture chamber, in which a planar carrier for the cell culture from a flexible material formed as a band from a membrane or a tissue is wound up to form a spiral and is fastened to a holding element from a dimensionally stable material. The cell culture chamber has to be separated, on the one hand, from a supply chamber provided for receiving the supply medium by a semipermeable membrane and, on the other hand, from the surrounding atmosphere by a gas exchange membrane which is impermeable to liquids but permeable to gases. This cell cultivation vessel is to be easy to handle and inexpensive to produce and to enable a cell culture process with which high cell densities can be achieved.

DE 10 2010 005 415 A1 relates to a device with which stem cells growing adherently or in suspension and primary cells from human or animal sources can be expanded dynamically and continuously in perfusion mode in a rotating bed bioreactor. This device for the cultivation, propagation and/or differentiation of primary cells or stem cells suspended in culture media consists of a reactor vessel with a removable closure cover, adjustable connections for the inflow and outflow of media, connections for the inflow and outflow of overlay atmosphere and a rotating shaft arranged axially in the reactor vessel and driven in a contactless manner via a magnetic drive. One or more rotating beds consisting of cell carriers are arranged on the shaft and are moved alternately through a culture medium and through the overlay atmosphere present in the head space of the reactor vessel by way of the contactless magnetic drive. The rotatable shaft has to be mounted detachably in the reactor vessel on one side and on the other side of the reactor vessel in a cover which can be removed from the reactor vessel. The rotating bed has to consist of a stack of cell carriers arranged one above the other, at a defined distance from one another, parallel to the shaft of the rotating bed in the form of rectangular disks of different size, in each case with a width adapted to the diameter of the reactor vessel and a thickness of 0.2 mm to 2 mm, and round end disks with correspondingly dimensioned grooves for fixing the rectangular disks.

EP 3 071 040 B1 describes an in vitro method for producing a cultivated meat product, comprising i) modifying a self-renewing cell line of farm animal, poultry, wild or aquatic animal species with an inducible myogenic transcription factor in order to produce a cell line modified with a myogenic transcription factor, wherein the self-renewing cell line is a pluripotent embryonic stem cell line or an induced pluripotent stem cell line, ii) keeping the modified cell line in a self-renewing process and subsequently inducing a myogenic differentiation of the modified cell line by exogenous regulation of the inducible myogenic transcription factor, wherein the inducing of the myogenic differentiation further comprises bringing the modified cell line into contact with a DNA methylation inhibitor and wherein the differentiated modified cell line forms myocytes and multinuclear myotubes, which each comprise myonuclei, and iii) cultivating the myocytes and the multinuclear myotubes in order to produce a cultivated meat product. This method is intended to succeed in the scalable in vitro cultivation of meat from a self-renewing source for dietary nutrition.

WO 2019/211189 A1 discloses a device with an elongated body for producing tissue from cells. The elongated body has to have at least one circumferential groove and extends substantially flush centrally through a trough extending in a closed path, wherein at least one of the circumferential grooves opens into an inner edge of the trough. This device is intended to make artificial muscle meat accessible with an automated and scalable method.

CA 2 566 841 C is directed to a bioreactor comprising a membrane support structure and a highly porous membrane which rests on the membrane support structure. The membrane has a nutrient side and a so-called gas side. Furthermore, the membrane has to contain an immobilized biolayer. The membrane has to be arranged and adapted such that it enables the diffusion of a nutrient solution from the nutrient side to the immobilized biolayer without external pressure. Finally, the membranes have to be arranged in pairs on the membrane support structure and to define an inner region. The bioreactor of CA 2 566 841 C is to be inexpensive and durable and to enable higher bioconversion rates than conventional systems.

From CA 3 086 283 A1, a biorcactor for cultivating cells is known, comprising a base part with a first chamber containing a stirrer and a first central column which is removably attached to the base part, wherein the first central column forms at least a part of a second, outer chamber for cultivating cells and a third inner chamber for returning the fluid flow from the second outer chamber to the first chamber. In this way, a bioreactor of modular construction is to be provided, with which one or more structured fixed beds can be used in order to simplify the production process and at the same time to achieve excellent cell cultivation results in terms of homogeneity and repeatability.

EP 3 068 866 B1 discloses a system for cell expansion, containing a bioreactor, a drive for rotating the bioreactor, a fluid circulation unit which is fluidically connected to the bioreactor, a pump for circulating fluid through the fluid circulation unit and the bioreactor, a processor and a memory which stores the processor-controlled instructions. With this system, cells in a bioreactor which is connected to a cell expansion system are to be able to be loaded and distributed without problems.

WO 2020/163329 A1 relates to a cell culture matrix containing a substrate, comprising a first side, an opposite second side and a multiplicity of openings which are formed in the substrate and which pass through the substrate. In this case, the multiplicity of openings has to be configured such that it permits the throughflow of a cell culture medium, of cells or of cell products. The technical teaching of WO 2020/163329 A1 is intended to enable cell culture matrices for the cultivation of cells with high density, uniform cell distribution and increased harvest yields.

### SUMMARY

The methods for producing and obtaining cultivated meat are always very complex and generally not suitable for production on an industrial scale. Thus, there is accordingly a need of making available methods or devices for the production of cultivated meat which are no longer afflicted with the disadvantages of the prior art and which can be used in some cases in an uncomplicated manner and reliably ensure a high yield. In some cases, the present disclosure was based on the object of making available devices for the production of cultivated meat which enable sustainable management.

Accordingly, a device for propagating cells such as differentiated and undifferentiated cells, in some cases differentiated cells, has been found, comprising a housing, in some cases having a cylindrical casing, with a longitudinal orientation extending from a first housing end to a second housing end, a plastic film present in the housing wound up in a spiral shape around a winding axis oriented along or parallel to the longitudinal orientation, with a first and a second film end and opposite side edges and with an inner and an opposite outer side, wherein on the inner side or on the outer side or on the inner and the outer side of the plastic film, in some cases on the inner or the outer side of the plastic film, a multiplicity of spacers is present, arranged and adapted to keep the inner side of the wound up plastic film spaced apart from the outer side of in each case adjacent film sections in the wound up state at least in sections, in some cases completely, such that a liquid medium can be transferred along the longitudinal orientation, in some cases from the first housing end to the second housing end.

### BRIEF DESCRIPTION OF THE DRAWINGS

Further features and advantages of the present disclosure result from the following description, in which expedient embodiments of the present disclosure are explained by way of example with reference to schematic drawings. In the figures:

FIG. 1 shows a schematic perspective representation of the device according to the present disclosure;

FIG. 2 shows a schematic cross-sectional view through the device according to the present disclosure according to FIG. 1;

FIG. 3 shows a schematic representation of a detail of the device according to the present disclosure according to FIG. 1;

FIG. 4 shows a schematic representation of a further embodiment of the device according to the present disclosure;

FIG. 5 shows a schematic representation of a further embodiment of the device according to the present disclosure;

FIG. 6 shows a schematic representation of a detail of the device according to the present disclosure according to FIG. 5; and

FIG. 7 shows a schematic representation of a further embodiment of the device according to the present disclosure.

### DETAILED DESCRIPTION

Suitable cells to be cultivated are generally primary cells of animal or human origin, wherein the device according to the present disclosure is in some cases suitable and intended for the propagation of muscle cells as primary cells. With the cells to be cultivated, cultivated food is accessible with the aid of the device according to the present disclosure. Cultivated food should in this case comprise cultivated meat as well as cultivated fish and cultivated seafood, wherein cultivated meat also comprises cultivated poultry meat.

The housing is expediently liquid-tight and in some cases also gas-tight. In this way, it can be ensured that no nutrient liquid escapes during the operation of the device according to the present disclosure. The tightness of the housing also contributes to the fact that with the device according to the present disclosure the cell propagation as well as the harvesting of the propagated cells can take place under axenic conditions.

The device according to the present disclosure can in some cases be equipped with a shaft. This shaft is in some cases oriented in the housing along or parallel to the longitudinal orientation thereof. The shaft can be rotatable as well as stationary. However, it has proven advantageous for many applications to use a rotatable shaft. The shaft is in some cases connected to a film end, in some cases the first film end, of the plastic film.

In some cases for the case where the cells propagated on the plastic film are to be mechanically harvested, use is made of a housing, in some cases comprising the cylindrical casing, which has an, in some cases a reversibly closable, opening gap in the housing or the casing with a first and an opposite second opening edge for the passage of the plastic film, which extends in some cases along the longitudinal orientation. The release of the cells adhering to the surface of the plastic film succeeds particularly well if the first opening edge or the second opening edge of the opening gap is formed as a scrapping edge for propagated cells adhering to the plastic film or is equipped with a scrapping edge for the propagated cells.

The plastic film is present in the housing wound up in a spiral shape. If use is made of a shaft in the device according to the present disclosure, the plastic film is present wound up on this shaft. In this case, the plastic film can be connected to the shaft, in some cases at its one end. In this way, the winding process is particularly flawless. Expediently, in some cases, the plastic film is detachably connected to the shaft. In this way, the complete removal of this film from the housing succeeds when the plastic film is pulled through an opening gap. If the propagated cells are mechanically removed when the plastic film is pulled out of the opening gap, an optimized harvesting can thus be ensured.

In some more expedient configurations, the shaft constitutes a hollow cylinder or is present in a hollow cylinder. In the latter case, the plastic film is present wound up on the hollow cylinder and is in some cases connected thereto, in some other cases at its one end. The shaft or the hollow cylinder, respectively, expediently has a diameter in the range from 5 to 40 cm, in some cases in the range from 8 to 25 cm.

A in some cases comfortable and in some other cases also reliable handling of the device according to the present disclosure also results from the fact that it is further equipped with a drive unit for the shaft. In this way, both the winding process and the unwinding process of the plastic film can be facilitated. In this way, axenic working in a permissible manner is also ensured. Manual interventions can be avoided in this way.

In a particularly suitable embodiment, the device according to the present disclosure further has a further shaft outside the housing. This further shaft can be used to pull out the plastic film emerging from the opening gap during the mechanical production of propagated cells. At the same time, in this process, the plastic film is wound up in a space-saving manner on the shaft present outside the housing. This shaft can be connected to a drive unit for the purpose of automation.

The housing of the device according to the present disclosure can further be equipped with a base plate. The housing can also have a fastening device for the shaft present in the housing or the hollow cylinder. In one embodiment, this fastening device can also be attached to the base plate. For the purpose of winding up and unwinding the plastic film on a shaft present in the housing, a drive unit connected thereto, for example an electric motor, can be provided. For many applications, it has proven expedient to configure the housing interior in an axenic manner and to ensure that the plastic film is introduced into the housing under axenic conditions.

For many applications, it has proven particularly advantageous for the spacers on the inner and/or the outer side, in some cases on the inner or the outer side, of the plastic film to extend in sections or completely along, in some cases parallel to, the longitudinal orientation. In this case, provision can be made for the spacers on the inner and/or, in some cases or, the outer side of the plastic film to extend from the first housing end or at a distance from the first housing end in the direction of or as far as the second housing end, in some cases from the first housing end as far as the opposite second housing end. In order that the mutually adjacent film layers of the wound up plastic film are not in contact with one another or come into contact with one another, respectively, with an even greater degree of certainty, provision can be made in one configuration for spacers adjacent to one another on the inner side or on the outer side to be in each case, in some cases uninterruptedly, of linear configuration, wherein this linear configuration extends along the longitudinal orientation.

In a particularly suitable embodiment, the spacers can be present in the form of an, in some cases tubular, air cushion film. In this case, the tubular air cushions can be formed in some cases by two plastic films connected to one another alternately in sections. These tubular air cushions in some cases extend along the longitudinal orientation of the housing and in some cases are present substantially parallel to one another. Alternatively, the spacers can be an integral constituent part of the plastic film. This can be brought about, for example, by the plastic film having correspondingly shaped embossings, for example obtained by thermal and/or mechanical manipulation of the plastic film. In order to arrive at spacers by way of embossing, use is in some cases made of plastic films composed of thermoplastic polymers. According to a further alternative configuration, the spacers can be formed from a plastic material, wherein use is expediently made of silicone compounds for this purpose. This can be present on the inner or on the outer side of the plastic film and in some cases also on both sides of the plastic film. The spacers manufactured from silicone compound can also be, in some cases, a multiplicity of drop-shaped or substantially round or circular elevations, respectively. Spacers formed in this way are of course also accessible by way of embossing forming, as described above.

In an expedient embodiment of the device according to the present disclosure, the spacers of adjacent wrapping layers or of mutually adjacent film portions of the plastic film wound up in a spiral shape, respectively, are arranged substantially congruently. Also in this way, it succeeds effectively that adjacent film layers are always spaced apart sufficiently far from one another over the entire circumference and leave space for the propagation of the cells and at the same time ensure the unhindered passage of the nutrient solution. Alternatively, the spacers of such adjacent film portions can also be arranged offset with respect to one another.

The spacers generally have an average height, in some cases absolute height, in the range from 1.0 to 15.0 mm, in some cases in the range from 1.5 to 10.0 mm and in some other cases in the range from 2.0 to 5.0 mm. The distance between mutually adjacent film web portions in the wound up film web is generally in the range from 0.3 to 10.0 mm, in some cases in the range from 1.0 to 5.0 mm and in some other cases in the range from 1.5 to 4.0 mm.

The plastic film of the device according to the present disclosure can be intended for single use and also for multiple use. In both cases, it has proven particularly advantageous to resort to embodiment variants in which the plastic film and the spacer are formed from plastics of the same type. This makes it possible to feed this product to a recycling process, from which substantially identical products are then again accessible. For multiple use of the device according to the present disclosure or of components of this device, respectively, use can be made of established CIP or CIS protocols and methods, respectively (“clean-in-place” or “steam-in-place”). CIP protocols and methods for cleaning components of installations, such as are used, for example, in the food and pharmaceutical industry, are sufficiently known to the person skilled in the art. For the purpose of cleaning according to a CIP/CIS protocol, the production or processing process, respectively, is generally to be interrupted, and the installation is to be freed of all process materials.

The, in some cases coated or treated, plastic film of the device according to the present disclosure constitutes a growth surface. In order that cells adhere or grow, respectively, on the plastic film and can subsequently be propagated in the presence of a nutrient solution, it has proven advantageous to treat the inner side, the outer side or the inner and the outer side of the plastic film in such a way that suitable adherence options are offered to the cells. In many cases, this succeeds particularly well by imparting a certain polarity to a surface in any case. Accordingly, use can be made for the plastic film of those inner sides and/or outer sides which have been plasma-treated, corona-treated or alkali-treated.

It has also proven sufficient for many applications to provide the inner side, the outer side or the inner and the outer side of the plastic film with a surface roughness. In this case, those inner and outer sides are expedient which have an average surface roughness Sa in the range from 0.01 to 100μ, in some cases in the range from 0.1 to 75 μm and in some other cases in the range from 1.0 to 50 μm, in each case determined in accordance with DIN EN ISO 25178 (2010-2020).

Furthermore, a support which is particularly advantageous for adhering or propagating, respectively, on and cultivating cells can also be obtained by providing the inner side, the outer side or the inner and the outer side of the plastic film with a suitable coating. In this case, such coatings are expediently based on or formed from gelatin, poly-L-lysine, poly-D-lysine, poly-ornithines, collagen, in some cases collagen I, II or IV, fibronectin, laminin, elastin, entactin, vitronectin, osteopontin, matrigel, hydrogel, aginate gel, lactate gel and/or constituents of the basal membrane from Engelbreth-Holm-Swarm (EHS) mouse tumors.

Such coating, in some cases over the full area, have proven to be highly expedient which are based on thermo-responsive material for the inner side, the outer side or the inner and the outer side of the plastic film. Accordingly, provision can be made for the surface of the inner side, the outer side or the inner and the outer side of the plastic film to be equipped at least in sections, in some cases substantially completely, in some other cases completely, with a thermos-responsive material or for the plastic film to comprise or consist of a thermos-responsive material.

The plastic film can be formed from one or more layers. In one embodiment, the plastic film can be a coextrusion film containing, for example, 3, 5 or 7 individual layers.

With the device according to the present disclosure, diverse applications can be realized. Thus, the device according to the present disclosure can be adapted, on the one hand, for applications on a laboratory scale and, on the other hand, for the production of cultivated food such as cultivated meat on an industrial scale. Accordingly, the plastic film can have a length in the range from 2 to 1000 m. Alternatively and in some cases additionally, provision can be made here for the width of the plastic film to be in the range from 0.25 to 2.0 m. For many applications, it has proven expedient to set the thickness of the plastic film such that it is in the range from 0.05 to 1.0 mm, in some cases in the range from 0.05 to 0.5 mm and in some other cases in the range from 0.1 to 0.3 mm.

The object on which the present disclosure is based is in some cases also achieved reliably and successfully by virtue of the fact that, in expedient embodiments, the plastic film has a flexural strength greater than or equal to 1000 mN*cm², in some cases greater than or equal to 2000 mN*cm², for example in the range from 1000 to 10000 mN*cm² or 2000 to 7500 mN*cm², in each case determined in accordance with DIN 53362:2003-10. In this way, it can in some cases also be ensured that even in the case of small gap widths between adjacent layers of the wound-up plastic film, said layers do not come into contact with one another by way of the spacers even in the case of relatively long periods of use of the device according to the present disclosure.

With the device according to the present disclosure, it is possible for the housing, the plastic film or the spacers, in some cases the housing, the plastic film and the spacers, to be based on polymers composed of renewable raw materials. Furthermore, provision can be made for the housing, the plastic film or the spacers to be based on polylactic acid, polyolefins, in some cases polyethylene or polypropylene, polyhydroxyalkanoates, polycaprolactones, polyesters, polyamides or starch, wherein use is in some cases made of polylactic acid.

In some more expedient embodiments, the device according to the present disclosure can have at least one inlet opening, in some cases in the region of the first housing end, and at least one outlet opening, in some cases in the region of the second housing end. Alternatively and in some cases additionally, the device according to the present disclosure can further have at least one feed line for the nutrient solution, in some cases in the region of the first housing end, and at least one discharge line for the nutrient solution, in some cases in the region of the second housing end. The side edges of the plastic film are expediently arranged adjacent to the first or second housing end. In this way, the volume of the housing can be utilized in the best possible manner in order to achieve a high harvest yield.

A particularly reliable cell propagation can in some cases also be obtained with such devices according to the present disclosure which have at least one temperature, oxygen, carbon dioxide, pH, lactate, fructose, glucose and/or, in some cases and, conductivity sensor, in some other cases at least one oxygen, lactate and glucose sensor, in some cases in the region of or in the feed line and/or in the region of or in the discharge line. Alternatively and in some cases additionally, provision can be made for at least one sensor to be provided, arranged and adapted for determining the flow rate of the nutrient solution in the feed line or adjacent to the feed line and/or, in some cases and, at least one sensor arranged and adapted for determining the flow rate of the nutrient solution in the discharge line or adjacent to the discharge line.

In order to maintain sterile and in some cases axenic working conditions in the housing, it has proven expedient for many applications to provide or integrate, respectively, at least one sterile filter in the feed or discharge lines, in some cases in the feed and discharge lines, which is provided for the transfer of gases. Furthermore, it is advantageous in this connection, alternatively and in some cases additionally, to provide or integrate, respectively, a sterile connector in the feed or discharge lines, in some cases in the feed and discharge lines, for the transfer of liquid or gaseous substances.

In a very expedient further development, the device according to the present disclosure also comprises a collection container connected or connectable to the discharge line for receiving nutrient solution flowing out of the housing. This collection container can in this case also comprise a stirring unit and/or a heating unit. Furthermore, it has proven advantageous if this container is or can be brought into operative connection with a gassing unit. This gassing unit or its feed line to the collection container, respectively, can also be equipped with at least one sterile filter. This container can accordingly serve as a conditioning vessel for the nutrient solution. Furthermore, an optimized mode of operation can in some cases also be ensured in that the collection container is equipped with a temperature, oxygen, carbon dioxide, pH, lactate, fructose and/or, in some cases and, conductivity sensor. With the aid of the collection container described, the nutrient solution removed via the discharge line can be prepared and fed back to the housing.

Once the cultivation of the cells is complete, the propagated cells adhering to the plastic film can be detached therefrom and obtained in diverse ways. For example, according to one embodiment, the plastic film can be pulled out through an opening gap present in the housing, wherein the propagated cells are mechanically released/scraped off along an edge or bead. For the purpose of facilitating the production of the released/scraped off cells, it has proven expedient to apply a liquid or gaseous medium to the opening gap or the edge or edges of the opening gap, respectively, in some cases via a nozzle arranged in the housing. The plastic film present wound up in the housing is unwound in this process. In addition to the mechanical recovery of the propagated cells, it has proven particularly advantageous to release these propagated cells physically, for example by changing the temperature, in some cases by incorporating thermos-responsive coating material, ultrasonic treatment or the action of light, or chemically, for example by treatment with trypsin. In expedient embodiments, the physical and the chemical release take place in the housing of the device according to the present disclosure. The released cells can then be discharged from the housing together with the nutrient solution and isolated.

The vessel for the nutrient solution, also referred to as conditioning vessel, can also be present in some cases including all feed and discharge lines inserted in the housing.

The conditioning vessel is in some cases configured such that it can also be operated with commercially available process control systems.

The present disclosure is accompanied by the surprising finding that an expedient concentration gradient can be established or maintained, respectively, with the device according to the present disclosure along the transport path of the nutrient solution. Consequently, a trouble-free passage of the nutrient solution is ensured with the device according to the present disclosure. Moreover, it is ensured with the device according to the present disclosure that the distances between adjacent film portions are sufficiently large for the passage of the nutrient solution, specifically even after advanced propagation of the cells adhering to the plastic film. A throughflow, in some cases a laminar throughflow, of the nutrient solution can be ensured and maintained with the device according to the present disclosure. A low-shear supply with nutrients succeeds reliably. As a result, a cultivation vessel for the expansion of adherent cells is made available with the device according to the present disclosure, with which cultivation vessel cultivated food, in some cases cultivated meat, cultivated fish or cultivated seafood, can be made available on an industrial scale reliably and in a consistently high quality. It is also of particular advantage in the case of the device according to the present disclosure that the housing can be reused after use has taken place. With the plastic film as a growth surface as a component of the device according to the present disclosure, the costs for the propagation or cultivation, respectively, of cells, in some cases differentiated cells, can be significantly reduced in comparison with methods of the generic type.

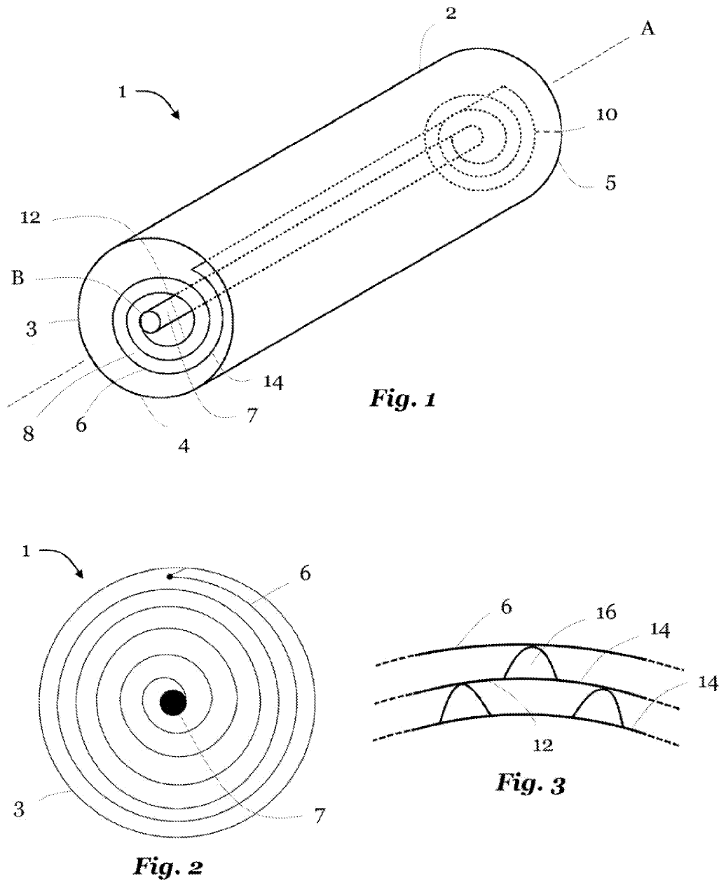

FIG. 1 shows a schematic perspective representation of a device (1) according to the present disclosure for propagating cells, for example differentiated cells. For a clearer representation, the spacers are not shown therein. In the represented embodiment, this device (1) comprises a housing (2) with a cylindrical casing (3). The housing (2) has a longitudinal orientation (A) extending from a first housing end (4) to a second housing end (5). A winding axis (B), which in the present case is embodied as a shaft (7), is oriented colinearly to this longitudinal orientation (A). A plastic film (6) wound up in a spiral shape is arranged around this shaft (7). This plastic film (6) is accordingly present in the housing (2). It is equipped with a first and a second film end (8, 10) and opposite side edges and with an inner and an opposite outer side (12, 14). FIG. 2 shows a schematic cross-sectional view through a device (1) according to FIG. 1. As can be derived from the detail of the device according to FIG. 1 reproduced in FIG. 3, a multiplicity of spacers (16) are present on the outer side (14) of the plastic film (6). These have a longitudinal orientation in the represented embodiment. These spacers (16) keep the inner side (12) spaced apart from the outer side (14) of the film portion adjacent in the wound up state in the wound up plastic film (6). In this way, liquid can be transferred along the longitudinal orientation from the first housing end (4) to the second housing end (5). The spacers (16) in some cases keep the inner side (12) spaced apart from the outer side (14) over all mutually adjacent wrapping layers in the wound up plastic film (6).

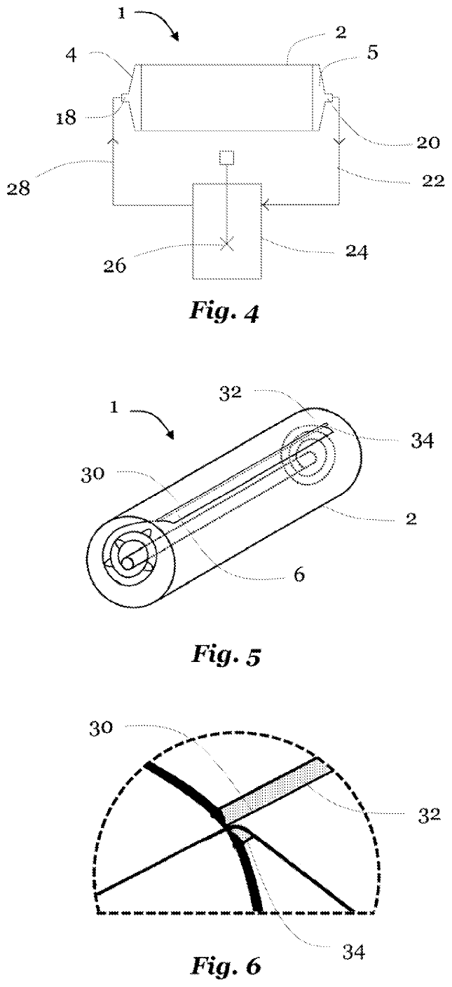

FIG. 4 shows a further, highly expedient embodiment of a device (1) according to the present disclosure, in which an inlet opening (18) is present in the region of the first housing end (4) and an outlet opening (20) is present in the region of the second housing end (5). A discharge line (22) is arranged at the outlet opening (20), via which discharge line nutrient solution can flow out of the housing (2) and can be received and collected in a collection container (24), for example in order to be heated therein and to be supplemented with fresh nutrient solution. In the represented variant, this collection container (16) is equipped with a stirring unit (26). The prepared nutrient solution can then be transferred via the feed line (28) to the inlet opening (18) arranged in the region of the first housing end (4) and introduced back into the housing (2) of the device.

FIG. 5 shows a further embodiment of the device (1) according to the present disclosure. In this embodiment, the cylindrical casing of the housing (2) is equipped with a reversibly closable opening gap (30) with a first and an opposite second opening edge (32, 34). The plastic film (6) can pass through this opening gap (30), which extends in the represented embodiment along the longitudinal orientation (A).

From FIG. 6 a detail of the opening gap (30) with the first and the second opening edge (32, 34) can be derived, as can be used in the device according to FIG. 5. In the represented embodiment, the first opening edge and the second opening edge of the opening gap are formed as scrapping edges for propagated cells adhering to the plastic film.

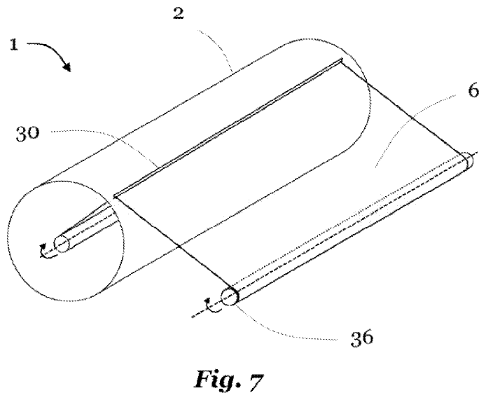

FIG. 7 shows a schematic representation of a further embodiment of the device (1) according to the present disclosure. This is equipped with a further shaft (36), which is present outside the housing (2) and with the aid of which the unwinding and winding up of the plastic film (6) emerging from the opening gap (30) can be automated.

The features of the present disclosure disclosed in the above description, in the claims and in the drawings can be essential both individually and in any desired combination for the realization of the present disclosure in its various embodiments.

What is claimed is:

1. A device for propagating cells comprising:
a housing with a longitudinal orientation extending from a first housing end to a second housing end;
a plastic film present in the housing, wound up in a spiral shape around a winding axis oriented along or parallel to the longitudinal orientation, with a first and a second film end and opposite side edges and with an inner and an opposite outer side,
wherein on the inner side or on the outer side or on the inner and the outer side of the plastic film a multiplicity of spacers is present, arranged and adapted to keep the inner side of the wound up plastic film spaced apart from the outer side in sections or substantially completely in the wound up state, such that a liquid medium can be transferred along the longitudinal orientation.

2. The device according to claim 1, further comprising a shaft oriented along or parallel to the longitudinal orientation, wherein the plastic film is present on the shaft wound up in a spiral shape.

3. The device according to claim 2, further comprising a drive unit for the shaft.

4. The device according to claim 1, wherein the housing has an opening gap with a first and an opposite second opening edge for the passage of the plastic film.

5. The device according to claim 1, further comprising at least one inlet opening and at least one outlet opening.

6. The device according to claim 1, further comprising at least one feed line for a nutrient solution and at least one discharge line for the nutrient solution.

7. The device according to claim 1, further comprising at least one temperature, oxygen, pH, lactate, fructose, glucose and/or conductivity sensor and/or at least one sensor arranged and adapted for determining the flow rate of the nutrient solution in the feed line or adjacent to the feed line and/or at least one sensor arranged and adapted for determining the flow rate of the nutrient solution in the discharge line or adjacent to the discharge line.

8. The device according to claim 1, further comprising a further shaft, present outside the housing, adapted and arranged for unwinding and winding up the plastic film emerging from the opening gap.

9. The device according to claim 1, wherein the inner side or the outer side or the inner and the outer side of the plastic film is plasma-treated, corona-treated or alkali-treated or has a coating based on or formed from gelatin, poly-L-lysine, poly-D-lysine, poly-ornithines, collagen, fibronectin, laminin, elastin, entactin, vitronectin, osteopontin, matrigel, hydrogel, aginate gel, lactate gel and/or constituents of the basal membrane from Engelbreth-Holm-Swarm (EHS) mouse tumors.

10. The device according to claim 1, wherein the inner side or the outer side or the inner and the outer side of the plastic film have a surface roughness.

11. The device according to claim 1, wherein the plastic film has a flexural strength greater than or equal to 1000 mN*cm2 determined in accordance with DIN 53362:2003-10.

12. The device according to claim 1, wherein:
the inner side or the outer side or the inner and the outer side of the plastic film are equipped at least in sections with a thermos-responsive surface; or
the plastic film comprises or consists of a thermos-responsive material; and/or
the spacers on the inner and/or the outer side of the plastic film extend in sections or completely along the longitudinal orientation; and/or
the spacers on the inner and/or the outer side of the plastic film extend from the first housing end or at a distance from the first housing end in the di-rection of or as far as the second housing end.

13. The device according to claim 1, wherein:
the spacers are present in the form of an air cushion film; and/or
the spacers are an integral constituent part of the plastic film; and/or
the spacers are present in the form of a plastic material on the inner side, the outer side or the inner and the outer side of the plastic film.

14. The device according to claim 1, wherein the spacers of adjacent wrapping layers are arranged substantially congruently.

15. The device according to claim 1, wherein spacers adjacent to one another on the inner side or on the outer side are in each case of linear configuration, wherein this linear configuration extends along the longitudinal orientation.

16. The device according to claim 4, wherein the first opening edge or the second opening edge of the opening gap is formed as a scrapping edge for propagated cells adhering to the plastic film or is equipped with a scrapping edge for the propagated cells.

17. The device according to claim 6, wherein at least one sterile filter is provided or integrated in the feed or discharge lines which are provided for the transfer of gases, and/or wherein at least one sterile connector is provided or integrated in the feed or discharge lines for the transfer of liquid or gaseous substances.

18. The device according to claim 1, further comprising a collection container connected or connectable to the discharge line for receiving nutrient solution flowing out of the housing.

19. The device according to claim 18, wherein the collection container comprises a stirring unit and/or a heating unit and is or can be brought into operative connection with a gassing unit if necessary.

20. The device according to claim 19, further comprising a connection to the feed line.

21. The device according to one of claim 18, further comprising a temperature, oxygen, carbon dioxide, pH, lactate, glucose, fructose and/or conductivity sensor.

22. Use of the device according to claim 1 for the production of cultivated food, cultivated fish or cultivated seafood, or for the recovery of propagated differentiated or undifferentiated cells.

23. The device according to claim 1, wherein the spacers are present on the inner or the outer side of the plastic film.

24. The device according to claim 2, wherein the shaft is rotatable.

25. The device according to claim 4, wherein the opening edge extends along the longitudinal orientation.

26. The device according to claim 10, wherein the surface roughness has an average surface roughness Sa in the range from 0.01 to 100 μm determined in accordance with DIN EN ISO 25178 (2010-2020).

27. The device according to claim 13, wherein the air cushion film is tubular.

28. The device according to claim 13, wherein the spacers are formed as film embossing.
